(19) United States
(12) Patent Application Publication
(10) Pub. No.: US 2012/0199602 A1
(43) Pub. Date: Aug. 9, 2012

(54) DISPENSER AND GLOVES

(76) Inventors: Earl Jordan, Laguna Woods, CA (US); Paul R. Persiani, Laguna Woods, CA (US)

(21) Appl. No.: 13/501,679

(22) PCT Filed: Oct. 12, 2010
(86) PCT No.: PCT/US10/52331

§ 371 Date: Apr. 12, 2012

Related U.S. Application Data
(60) Provisional application No. 61/251,158, filed on Oct. 13, 2009.

(51) Int. Cl. B65D 83/08; B65B 5/06; A61B 19/04; B65B 5/02

(52) U.S. Cl. 221/37; 53/452; 53/475; 221/45

(57) ABSTRACT

Gloves and dispensers for gloves are generally discussed herein with particular discussions extended to disposable gloves packaged in a disposable dispenser configured to engage with a holder. Aspects of the glove assemblies provided herein include a tray responsive to the number of gloves in the dispenser, movable in a vertical direction within the disposable dispenser towards the dispenser opening with its movement facilitated by a telescoping piston or other biasing members, such as a conical spring. The dispenser may be removed from the holder and a new dispenser engaged to the holder. Aspects of the present disclosure also include a disposable dispenser having a biasing member urging a tray in a vertical direction in response to the number of gloves in the dispenser.

### CROSS-REFERENCE TO RELATED APPLICATIONS

This is a national phase application under 35 U.S.C. §371 of PCT Application No. PCT/US2010/052331 filed Oct. 12, 2010, which claims the benefit of U.S. Provisional Application No. 61/251,158 filed Oct. 13, 2009, the contents of each of which are expressly incorporated herein by reference.

### FIELD OF ART

Gloves and dispensers for gloves are generally discussed herein with particular discussions extended to disposable gloves packaged in a dispenser for use with or without a holder.

### BACKGROUND

An unremitting upsurge in disposable glove use continues for over two decades. The initial surge occurred in 1985, when The Centers for Disease Control and Prevention (CDC) developed the strategy of “universal blood and body fluid precautions,” which are based on the premise that all patients should be assumed to be infectious for HIV and other blood-borne pathogens. These strategies were formalized in “CDC Guidelines for Prevention of Transmission of Human Immunodeficiency Virus and Hepatitis B Virus to Health-Care and Public-Safety Workers,” MMWR 1989; 38(S-6):1-36. In general. “universal precautions” require that disposable glove use be followed when workers are exposed to blood, certain other body fluids (amniotic fluid, pericardial fluid, peritoneal fluid, pleural fluid, synovial fluid, cerebrospinal fluid, semen, and vaginal secretions), or any body fluid visibly contaminated with blood. In recent years, many antibiotic-resistant, virulent, and lethal microorganisms have become increasingly widespread, including human immunodeficiency virus (HIV), hepatitis B, necrotizing staphylococcus, Vancomycin-resistant enterococcus and multi-drug-resistant tuberculosis. The use of gloves as a barrier to prevent transmission of microorganisms between patients and health care professionals has become a predominant issue in today's clinical settings, including physicians and dentists' offices.

Disposable glove use is burgeoning beyond the clinical setting as well. Emergency, law enforcement, correctional facility, and public-safety workers often encounter unpredictable and emergent exposures, which may make the identification of hazardous body fluids very difficult and often impossible. Furthermore, not only must workers be protected from exposure to blood and other potentially infectious body fluids in the course of their work activities, they also must protect others from infection through cross-contamination. For example, childcare and preschool centers, and even kindergartens, have become transmission points for antibiotic-resistant blood-borne pathogens.

Many workers in diverse occupations such as, in the electronics, medical device, pharmaceutical, agricultural, nuclear, industrial chemical and pesticide handling, waste management, painting, cosmetic, and body art industries and services increasingly look to a ready supply of disposable gloves to reduce their exposure to, and skin contact with, potentially hazardous materials. These materials can include toxic chemicals, pesticides, cytotoxic drugs, radioactive materials, and human, animal, or biological tissues, fluids, and wastes. Moreover, increased public concerns regarding the transmission of disease have led to measures to protect food during preparation and service.

Thus, disposable gloves have become the ubiquitous form of barrier-type personal protective equipment, creating a $1.4 billion latex and synthetic glove market in 2008, in which nearly 34 billion gloves were sold in the USA alone. In many settings, disposable gloves are either legally mandated or considered to be integral with the principles of good practice.

Disposable gloves should fit properly, and they should not be washed or reused. Also, gloves should be replaced once soiled or damaged, and in-between patients, victims, products, and work areas. It is desirable that even non-sterile disposable gloves be kept as clean as possible prior to use. Otherwise, biological and physical contaminants may be inadvertently transferred to the patient, the product, or the work area by the soiled gloves. Accordingly, it is most desirable to minimize contact with the finger portions of clean gloves.

Typically, disposable gloves are dispensed in random orientations from large containers or open plastic bins located at stationary points, which are subject to gross manipulation. These containers usually hold 100 or more gloves, are fixed to a sink, wall, or bulkhead, and may be covered by a metal, plastic, or wooden cover, primarily for cosmetic purposes. Often, these covers themselves can become reservoirs for pathogens. Also, these dispensers can be at some distance from the patients or work areas. If a worker exhausts his or her immediate supply of gloves, another trip to the dispenser site is required. As a result, glove users are often compelled to haphazardly cram extra gloves into pockets, pouches, and sacks, a practice that can grossly soil clean gloves, can intermingle clean objects with contaminated objects, and can lead to gloves falling out of pockets at inopportune moments.

Existing portable disposable glove holders do allow glove users to carry a ready supply of gloves. However, these portable disposable glove holders are reusable and refillable. With each subsequent reuse comes an additional risk of cross-contamination of the container, and thus the gloves, with accumulated microbes, chemical agents, dust, soil, blood, and any other particulate foreign material. Furthermore, such portable dispensers are refilled by manually cramming a quantity of randomly-oriented gloves, which first were plucked from a dispenser, such that gloves are handled by contact with whatever glove surface may be present at the dispenser opening, including the fingers.

### SUMMARY

The present apparatus, device, and method may be practiced by providing a dispenser adapted to store a bundle of gloves. The dispenser can comprise a dispenser housing comprising a plurality of sidewalls comprising a top flange, a top wall comprising an opening and a height along a longitudinal direction. A dispenser base can be included comprising a base wall having an opening. The sidewalls, the top wall and the base wall together define a receiving space for storing the bundle of gloves. A tray is positioned within the receiving space and adapted to move within the receiving space along the longitudinal direction relative to the dispenser base.

The present apparatus, device, and method may also be practiced by providing a holder adapted to engage a glove dispenser, the holder comprising a holder housing comprising an engagement tab for engaging a glove dispenser. The holder housing comprising a top wall comprising an opening and a holder base attached to the holder housing. The holder base and the holder housing defining an interior cavity. A telescoping piston is disposed in the interior cavity having at least one piston section and a biasing member for biasing the at least one piston section to move relative to the holder base to project through the opening on the holder housing.

In yet another aspect of the present apparatus, device, and method, there is provided a combination glove dispenser and holder comprising a dispenser case adapted to contain a glove bundle. The dispenser case comprising a dispenser top and a dispenser bottom defining a receiving space. A tray is located in the receiving space and movable within the receiving space for facilitating the dispensing of the glove bundle. The holder comprising a telescoping piston adapted to urge the tray to move towards the dispenser top. In an alternative embodiment, the dispenser is self-contained and can be dispensed by placing on a counter-top, a shelf, a desk, or a working station. In this alternative embodiment, the holder is part of the dispenser as the spring for biasing the tray is located within the walls of the dispenser.

The present apparatus, device, and method may also be practiced by providing a dispenser adapted to store a bundle of gloves, the dispenser comprising a dispenser housing comprising a plurality of side walls, a top wall comprising an opening and a height along a longitudinal direction, and a dispenser base comprising a base wall. The sidewalls, the top wall and the base wall together define a receiving space for storing the bundle of gloves. A tray is located in the receiving space and movable along the longitudinal direction within the receiving space and positioned between the dispenser base and the top wall. At least one biasing member for biasing the tray to move along the longitudinal direction relative to the dispenser base towards the top wall. The biasing member may be self-contained and be part of the dispenser or is part of a holder for pushing the tray through an opening at the bottom of the base wall of the dispenser.

In yet another aspect of the present apparatus, device, and method, there is provided a combination glove dispenser and gloves comprising a glove bundle having a predetermined number of gloves disposed in a dispenser housing. The dispenser housing comprising a dispenser top, a plurality of sidewalls and a base wall defining a receiving space. A tray is located in and movable within the receiving space and positioned between the base wall and the dispenser top. A biasing means for biasing the tray from the base wall towards the dispenser top for facilitating the dispensing of the glove bundle is also provided.

In yet another aspect of the present apparatus, device, and method, there is provided a method for forming a combination dispenser and plurality of gloves. The method comprising forming a dispenser body comprising a plurality of side walls and a top wall having a dispensing opening and an installation opening; placing a plurality of gloves through the installation opening into a cavity of the dispenser body; positioning a tray subjacent the plurality of gloves, the tray adapted to move within the cavity; and providing a biasing member for biasing the tray to move in a longitudinal direction towards the top wall and closing the installation opening with a base wall. The biasing member may be self-contained and be part of the dispenser or is part of a holder for pushing the tray through an opening at the bottom of the base wall of the dispenser.

Other aspects and variations of the glove assemblies summarized above are also contemplated and will be more fully understood when considered with respect to the following disclosure.

### BRIEF DESCRIPTION OF THE DRAWINGS

These and other features and advantages of the present invention will become appreciated as the same become better understood with reference to the specification, claims and appended drawings wherein:

FIG. 1 is an exemplary exploded perspective view of a dispenser provided in accordance with aspects of the present disclosure;

FIG. 2A is an exemplary top view of the dispenser of FIG. 1 in an assembled state;

FIG. 2B is an illustration of a cross-sectional side view of the dispenser of FIG. 2A taken along line C-C;

FIG. 3 is an exemplary exploded perspective view of a holder provided in accordance with aspects of the present disclosure;

FIG. 4A is an exemplary top view of the holder of FIG. 3 in an assembled state;

FIG. 4B is an illustration of a cross-sectional side view of the holder of FIG. 4A taken along line D-D;

FIG. 5A is an exemplary top view of a dispenser and holder assembly;

FIG. 5B is an illustration of a side view of the dispenser and holder assembly of FIG. 5A;

FIG. 6A is an illustration of a top view of the dispenser and holder assembly when the dispenser is filled with gloves (not shown);

FIG. 6B is an illustration of a cross-sectional side view of the dispenser and holder assembly of FIG. 6A taken along line A-A;

FIG. 7A is an illustration of a top view of the dispenser and holder assembly when the dispenser is empty;

FIG. 7B is an illustration of a cross-sectional side view of the dispenser and holder assembly of FIG. 7A taken along line B-B;

FIG. 8 is an illustration of an exemplary exploded perspective view of another embodiment of the dispenser provided in accordance with aspects of the present disclosure;

FIG. 9 is an exemplary semi-schematic perspective view of a mounting bracket configured for receiving the dispenser of FIG. 8;

FIG. 10A is an exemplary top view of the dispenser of FIG. 8 in an assembled state; when the dispenser is full;

FIG. 10B is an illustration of a cross-sectional side view of the dispenser of FIG. 10A taken along line A-A;

FIG. 11 is an illustration of a cross-sectional side view of the holder in a nearly empty or empty state, with a few or without any gloves;

FIG. 12 is an exemplary perspective view of a dispensing package; and

FIG. 13 is an exemplary semi-schematic perspective view of a mounting bracket configured for receiving the dispensing package of FIG. 12.

### DETAILED DESCRIPTION

The detailed description set forth below in connection with the appended drawings is intended as a description of the presently preferred embodiments of disposable gloves, glove dispensers, and glove holders (hereinafter collectively referred to as “glove assembly or assemblies”) provided in accordance with aspects of the present invention and is not intended to represent the only forms in which the present invention may be constructed or utilized. The description sets forth the features and the steps for constructing and using the glove assemblies of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features

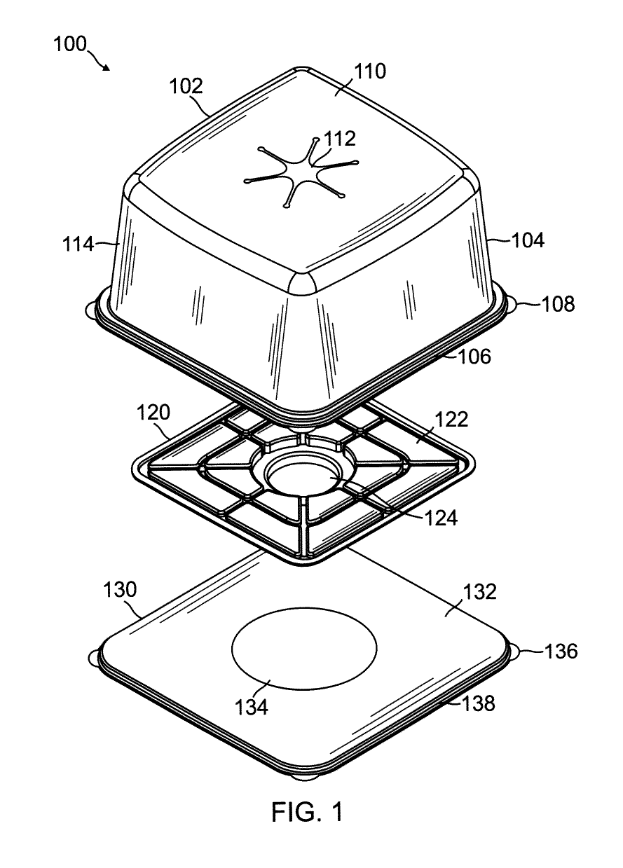

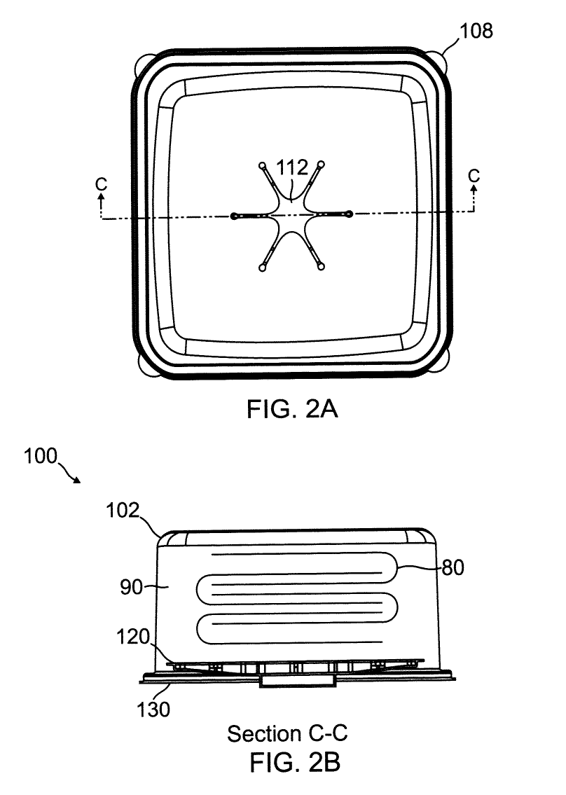

FIGS. 1, 2A, and 2B illustrate an embodiment of a glove dispenser 100 provided in accordance with the present disclosure. In one example, the dispenser 100 is configured to enclose a plurality of gloves folded by the methods disclosed in U.S. Pat. No. 6,901,723, the contents of which are expressly incorporated herein by reference. However, any folding technique may be used to fold the plurality of gloves into a stack of gloves aside from the superjacent opposing fold methods disclosed in the '723 patent. For example, the gloves may be folded in halves lengthwise but not widthwise prior to putting them in the superjacent opposing fold format, folded in halves and stacking them on top of one another, or folded in fourths and stacking them on top of one another, either with or without interleaving them, before placing them inside the glove dispenser 100. In a specific example, the plurality of gloves are folded in halves lengthwise in superjacent opposing fold format. When the methods described in the '723 patent are employed, the cuff section of a glove automatically projects out through the opening of the dispenser as the preceding glove is dispensed, as described in the '723 patent. In one embodiment, fifty individual gloves, i.e., 25 pairs, are folded and placed inside the dispenser. In other embodiments, more or fewer pair of gloves are placed inside the dispenser.

The dispenser 100 provided in accordance with aspects of the present disclosure may be made from a transparent or opaque plastic or other rigid or semi-rigid materials. The dispenser 100 can have a rectangular, a square, a cylindrical or any other appropriately shaped container configuration for storing gloves or bundles of gloves with rectangular and square configurations being more preferred.

FIG. 1 is a perspective view of an embodiment of the dispenser 100 provided in accordance with aspects of the present disclosure. The dispenser 100 comprises a dispenser housing 102 that can be made from any suitable natural, synthetic, composite, and laminate materials, including, without limitation, paper and board materials; metals and foils; rubber products; and plastics such as cellulose acetate butyrate (CAB), polystyrene, polyethylene terephthalate (PETG); polycarbonate; polyethylene, including PET, HDPE, and LDPE; polytetrafloralethylene; acetal, acrylic and combinations thereof. In a particular example, the dispenser housing 102 is made from a polyethylene terephthalate (PETG) material. In another example, the housing is made from a semi-rigid transparent thermoplastic material.

The dispenser housing 102 comprises a housing casing 104, which can include a top wall 110, a plurality of sidewalls 114, and a dispensing opening 112 disposed on the top wall 110. In one exemplary embodiment, the housing casing 104 is made from a vacuum formed process and the sidewalls 114 and top wall 110 are integrally formed from a single sheet of PETG or other suitable material comprising the opening 112.

In one exemplary embodiment, the dispensing opening 112, which is shown with a star like configuration, is located at the center of the top wall 110. However, it may be located on any appropriate side or portion of the dispenser 100, and in different opening configurations; for example an elliptical, curvilinear, rectilinear, or other appropriate shape without deviating from the spirit and scope of the present invention. A glove can be removed from the dispenser 100 by a user through the dispensing opening 112.

The dispenser 100 further comprises a dispenser base 130. The base 130 comprises a base wall 132 having an opening 134. Together, the top wall 110, the sidewalls 114 and the base wall 132 define a container cavity 90 (FIG. 2B) for receiving a plurality of folded gloves, as further discussed below. The cavity 90 may be sized to contain or hold any number of gloves, such as 25 pairs of gloves with more or fewer number of pairs contemplated.

The dispenser base 130 and the dispenser top 102 each has a peripheral flange 138 and 106, respectively, that generally overlap one another. After filling the container cavity 90 of the housing casing 104 with a stack of gloves 80 (FIG. 2B), the dispenser base 130 is mated with the dispenser top 102 and the two flanges 106 and 138 aligned. The two flanges are then bonded to one another to form a sealed dispenser 100. In one example, the two flanges 106 and 138 are welded together, using for example. e.g., a high frequency weld, laser, or other heating means, to more permanently seal the bundle of gloves placed inside the container cavity.

In one example, the dispenser top 102 comprises locking tabs 108 extending from the flange 106. In other embodiments, a plurality of locking tabs can be located anywhere along the flange 106 and can have a triangular, rectangular, or any other appropriate shape with semicircular being more preferred. Preferably, the locking tabs 108 are located at defined corners of the sealed flanges 106, 138. The locking tabs 108 are joined with the bottom tabs 136 that extend from the bottom flange 138 when the dispenser top is bonded to the dispenser base 130 to form a sealed dispenser 100. These locking tabs are incorporated to enable the dispenser 100 to engage a holder 200 for holding the dispenser 100 in a desired dispensing location, as further discussed below. As shown, the dispenser base 130 comprises an opening 134 for retaining a biasing element, which will further be described below in connection with the holder 200.

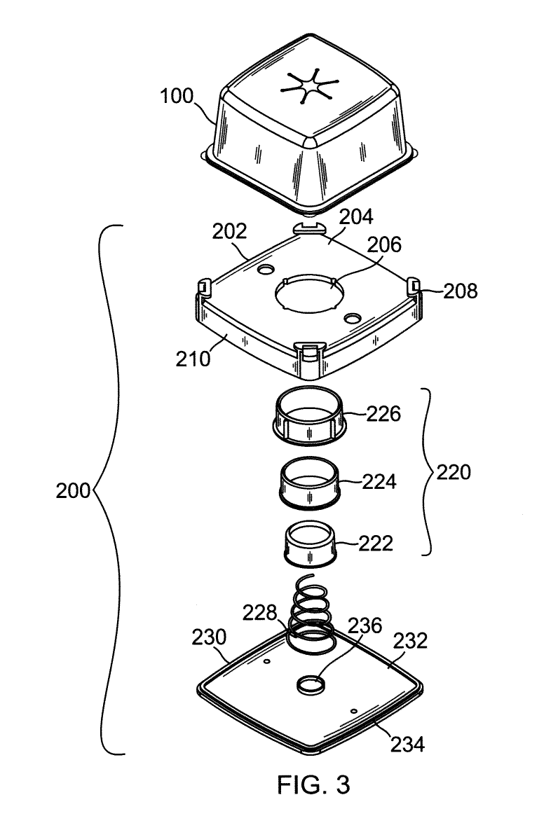

In one embodiment, the dispenser 100 further comprises a tray 120 located beneath the stack of gloves within the cavity 90 defined by the top wall 110, sidewalls 114 and base wall 132. The tray 120 is movable in a longitudinal direction from the dispenser base 130 towards the dispenser opening 112 in response to the number of remaining gloves inside the container and with the aid of a telescoping piston, as further discussed below. The tray 120 comprises ribs 122 and a contact plate 124, which is sized to project through the opening 134 on the dispenser base 130. In an alternative embodiment, the tray 120 simply overlaps and covers the opening 134 without projecting through the opening. The contact plate 124 is configured to contact and be pushed by a telescosping piston 220 located on the holder 200 (FIG. 3) through the opening 134 of the dispenser base 130. The telescosping piston 220 is configured bias the stack of gloves toward the dispenser opening 112. The amount or extent of movement of the tray 120 in a vertical or longitudinal direction can be proportional to the pressure from the stack of gloves within the dispenser 100. Generally speaking, the telescoping piston exerts a biasing force on the contact plate 124, which moves the tray 120 vertically depending on the number of gloves that remain in the dispenser 100. This in turn allows the next glove in the stack of gloves to be presented at the dispensing opening 112 for easy access by a user without the user having to reach inside the dispensing opening 112 for the next glove as the stack of gloves is reduced from usage. Thus, an aspect of the present device, apparatus, and method is a glove dispenser comprising a housing, a base, and a plurality of gloves located inside an interior cavity defined by the housing and the base. In a specific example, a tray is located adjacent the base and movable relative to the base to lift the plurality of gloves towards an opening on the housing.

FIG. 2A is a top view of the dispenser 100 shown in FIG. 1. FIG. 2B is a cross-sectional side view of the dispenser 100 of FIG. 2A taken along line C-C. In one embodiment, a removable tab (not shown) is placed over the dispensing opening 112 to seal the opening during packaging, shipping, and/or storage. With the dispensing opening 112 sealed by a removable tab, the base 130 sealed to the dispenser housing 102, and the contact plate 124 of the tray 120 occupying the opening 134 on the base, the dispenser 100 forms an enclosed environment suitable for commercial transaction, such as sanitary storage and packing. For clarity, the stack of gloves 80 folded in superjacent opposing fold format is shown schematically only, although it is understood to be included in the interior cavity 90 of the dispenser 100 and folded as shown in the '723 patent.

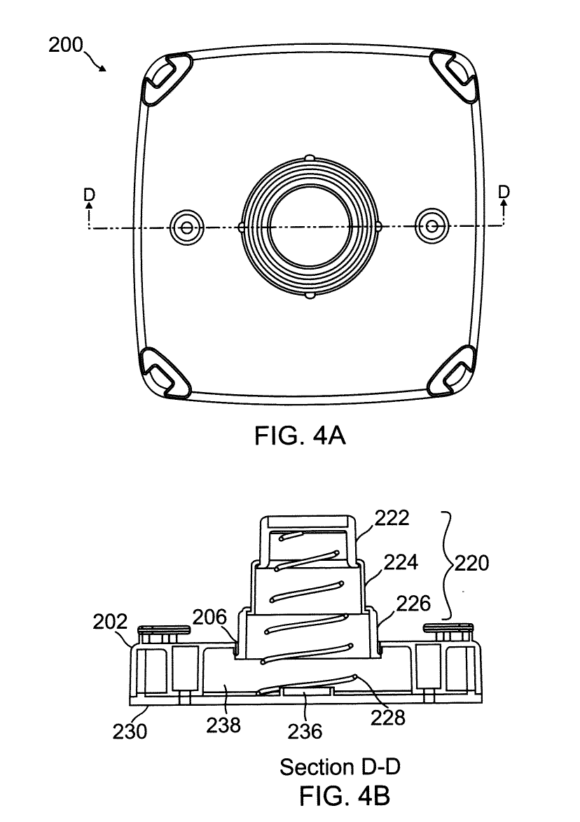

FIG. 3 shows a perspective view of a holder 200 for engaging the dispenser 100 according to aspects of the present apparatus, device, and method. FIG. 4A shows a top view of the holder 200 and FIG. 4B shows a cross-sectional side view of the holder 200 of FIG. 4A taken along line D-D. In one exemplary embodiment, the holder 200 is made from a rigid or semi-rigid thermoplastic material, with rigid being more preferred. Exemplary thermoplastic materials include suitable natural, synthetic, composite, and laminate materials, including, without limitation, polypropylene; polyvinylchloride; polycarbonate; polyethylene, including PET, HDPE, and LDPE; polytetrafloralethylene; and combinations thereof. Alternatively, the holder 200 may be made from paper and board materials; from a metal material (such as aluminum or stainless steel), from foils; from rubber products, or from combinations thereof with plastic being most preferred. The holder 200 can include attachment means allowing it to be mountable or portable. For example, through bores for fastening the holder to a surface or a workstation may be incorporated. As shown in FIG. 3, the holder 200 comprises a holder housing 202, a telescoping piston 220 and a holder base 230.

The holder housing 202 comprises a top wall 204 and sidewalls 210. The top wall 204 comprises an opening 206 and engagement tabs 208 for engaging the locking tabs of the dispenser 100, as further discussed below. The holder base 230 comprises a base wall 232, which along with the top wall 204 and sidewalls 210 of the holder housing 202 provide a cavity 238 (FIG. 4B) to accommodate the telescoping piston 220 when the piston is compressed, such as when the dispenser housing is full with a stack of gloves. The holder base 230 further comprises a flange 234 and an anchor point 236 for locating or anchoring the telescoping piston 220 within the cavity 238. In one example, the anchor point 236 is a projection, a post, or a pin for maintaining the position of the telescoping piston and the spring thereabout, as further discussed below.

In one embodiment, the telescoping piston 220 comprises multiple piston sections 222, 224 and 226 and a biasing member 228, which can be a spring, a shaped memory foam, a leaf spring, or a low durometer elastomer. Preferably, the biasing member is a helical spring. In another embodiment, one or more collapsible type material is used instead of multiple piston sections to compress and expand. For example, the collapsible type material can be an accordion type plastic or fabric material. When assembled, as shown in FIG. 4B, the telescoping piston 220 is urged by the biasing member 228 and projects out of the opening 206 of the holder housing 202. The projection of the telescoping piston 220 and the biasing force of the spring 228 is configured to urge the plate 124 on the tray 120 of the dispenser 100 (See, e.g., FIG. 2B) upwardly to then push the stack of gloves upwardly towards the dispensing opening 112 of the dispenser 100. The upward push on the tray is counter-acted, at least in part, by the pressure exerted by the stack gloves 80 located on the tray 120.

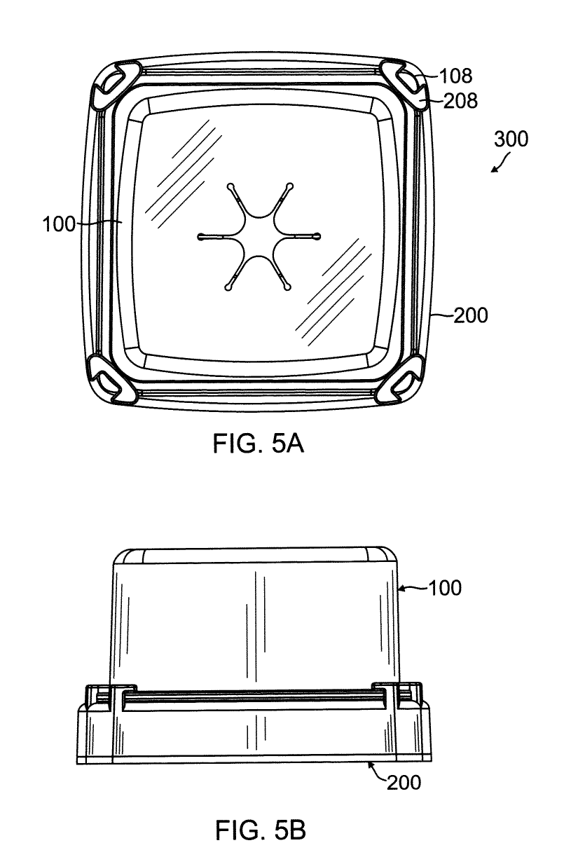

FIGS. 5A and 5B show a top view and side view of the dispenser holder assembly 300 according to aspects of the present disclosure. The assembly 300 comprises a dispenser 100 and a holder 200. As set forth above, the holder 200 has attachment means allowing it to be mountable on a surface, such as fastening holes for inserting mounting screws therethrough, or a clip for wearing on an article worn by a user, similar to clips for cell phones. The dispenser 100, which contains a stack of disposable gloves, is engageable with the holder 200 such that engagement tabs 208 of the holder housing 202 mechanically engage the locking tabs 108, 136 and keeps the dispenser 100 firmly in place. For example, the dispenser 100 may be placed against the holder 200 so that the locking tabs 108, 136 are located adjacent corresponding engagement tabs and then the dispenser 100 rotated to engage the attachment means. When so assembled, the tray 120 of the dispenser 100 is communicable with the telescoping piston 220, which projects through the opening 206 of the holder housing 202 (FIG. 3) and the opening 134 of the dispenser base 130 (FIG. 1). The telescoping piston 220 urges the tray 120 in an upward position to move the gloves toward the dispenser opening 112.

In another embodiment, straps or laces are used to hold the dispenser 100 against the holder 200. For example, the straps may be anchored or tied at one end to the holder 200, swung over the dispenser 100, and anchor at the other end to the holder 200. VELCRO may also be used to hold the dispenser 100 to the holder 200.

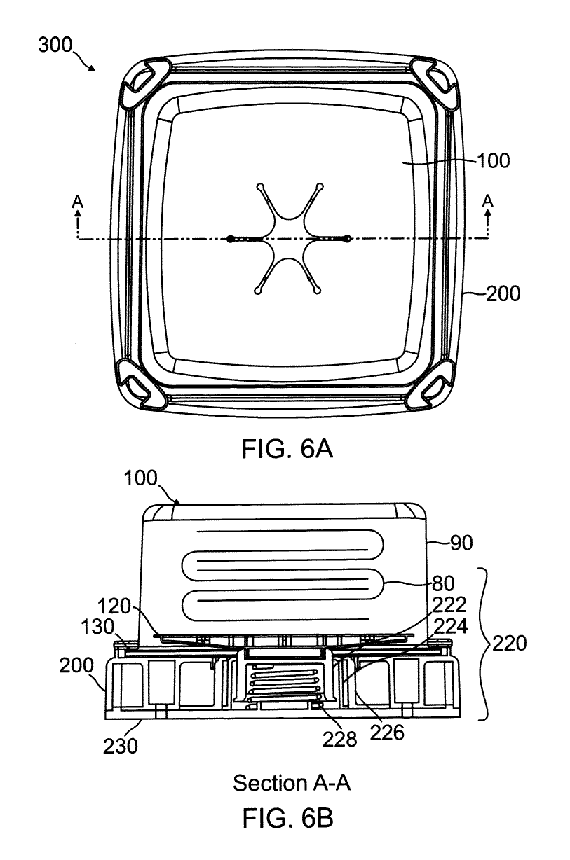

FIG. 6A shows a top view of the dispenser holder assembly 300 with the dispenser 100 comprising a stack of gloves 80. FIG. 6B shows a cross-sectional side view of the assembly 300 of FIG. 6A taken along line A-A with a stack of gloves 80 shown in schematic. The stack of gloves takes up most of the receiving space within the dispenser 100, forcing the tray 120 against the dispenser base 130. In this configuration, the telescoping piston 220 remains in a collapsed state due to the pressure and physical presence of the stack of gloves 80 located on top of the tray 120. As the gloves are dispensed, the stack of gloves gets lighter and smaller in size. In response to this reduced sized and pressure from the stack of gloves, the telescoping piston 220 expands and urges the tray 120 and the stack of remaining gloves upwardly towards the dispensing opening 112. Thus, regardless whether the dispenser is full or only partially full, the gloves are always easily accessible at the dispensing opening 112 by the urging capability of the holder.

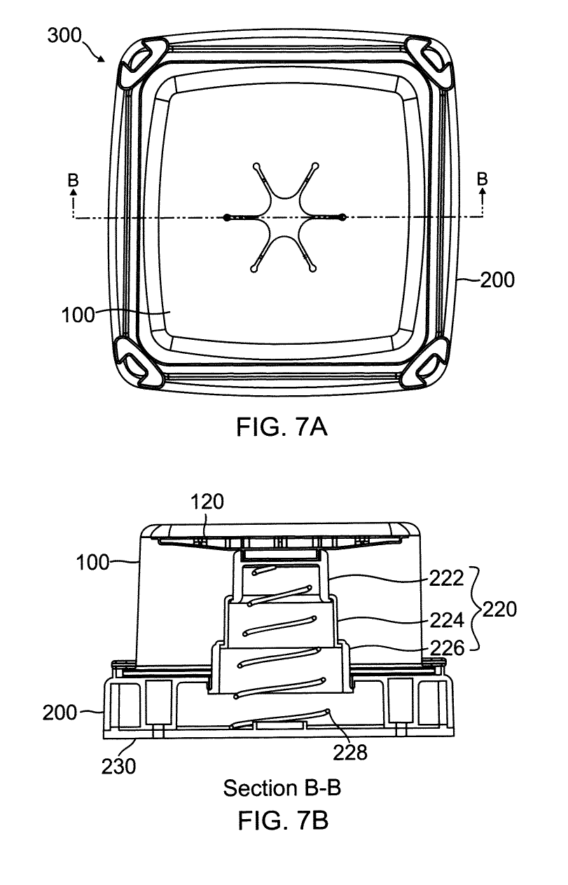

FIG. 7A shows a top view of the dispenser holder assembly 300 when the dispenser 100 is empty. FIG. 7B shows a cross-sectional side view of the assembly 300 of FIG. 7A taken along line B-B. Now that the stack of gloves is used up, i.e., the dispenser is empty, and the counter-pressure normally provided by the gloves being zero, the telescoping piston 120 fully expands and urges the tray 120 towards the dispenser opening 112. In one embodiment, the tray 120 is forced into contact with the top wall when the dispenser is empty.

The empty dispenser can now be removed and replaced. The dispenser 100 is removable from the holder 200 by disengaging the locking tabs 108, 136 from the corresponding engagement tabs 208. In one embodiment, this is accomplished by rotating the dispenser clockwise or counter-clockwise relative to the holder to separate the tabs from the engagement tabs. The dispenser can then be lifted away from the holder. A different dispenser 100 containing a plurality of stacked gloves may then be mounted to the holder 200 in the same manner as described above, or in reverse order from how the dispenser is removed as described immediately above. In other words, the holder 200 may be reusable while the dispenser 100 may be disposable when emptied. Obviously, a reusable dispenser case 100 may also be used with the holder 200 by incorporating means for opening the dispenser to refill it with additional gloves.

Accordingly, an aspect of the present disclosure includes a holder comprising a biasing element for urging a bundle gloves towards an opening on a dispenser. A further aspect of the present disclosure is a dispenser comprising a housing for containing a plurality of gloves having a movable tray that moves relative to the housing so that the tray, which has the plurality of gloves located therein, can be urged toward a dispenser opening on the housing. A still further aspect of the present disclosure is a combination dispenser and holder in which a biasing element is operative to move a tray of a dispenser to urge a bundle of gloves in a direction of a dispenser opening. In a specific example, the biasing element forms part of the holder. In yet another example, the biasing element is a combination helical spring and telescoping element. However, the biasing element can operate with or without the telescoping element.

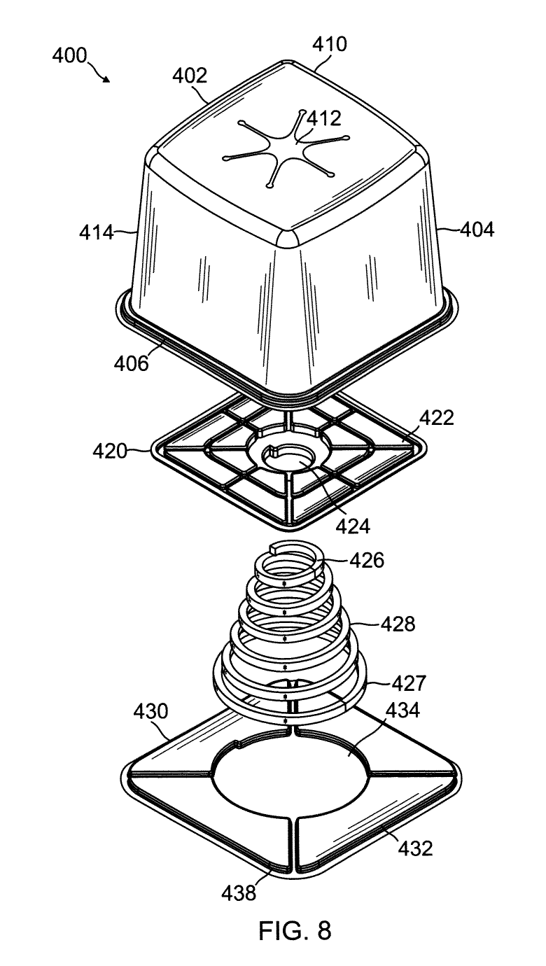

FIGS. 8, 10A, 10B, and 11 illustrate another embodiment of a glove dispenser 400 provided in accordance with aspects of the present apparatus, device, and method. In one exemplary embodiment, the dispenser 400 is configured to enclose a plurality of gloves folded by the methods disclosed in U.S. Pat. No. 6,901,723. However, any folding technique may be used to fold the plurality of gloves into a stack of gloves aside from the superjacent opposing fold methods disclosed in the '723 patent. In one specific example, the gloves are folded using the superjacent opposing fold methods in which the gloves are folded lengthwise only to reduce their lengths but not widthwise to reduce their widths.

The dispenser 400 provided in accordance with aspects of the present disclosure may be made from a transparent or opaque plastic or other rigid or semi-rigid materials. The dispenser 400 can have a rectangular, a square, a cylindrical or any other appropriately shaped container configuration for storing gloves or a bundle of gloves. In a preferred embodiment, the dispenser 400 has a sided configuration, such as a square or a rectangular configuration as opposed to being semi-spherical. In a less preferred embodiment, the dispenser is semi-spherical.

FIG. 8 is a perspective view of an embodiment of the dispenser 400 provided in accordance with aspects of the present disclosure. The dispenser 400 comprises a dispenser housing 402 made from a semi-rigid transparent thermoplastic material, such as from polyethylene terephthalate (PETG) or PVC material. In alternative embodiments, the dispenser 400 may be made from cardboard material. As shown, the dispenser housing 402 comprises a housing casing 404, which can include a top wall 410, a plurality of sidewalls 414, and a dispenser opening 412 disposed on the top wall 410. In one exemplary embodiment, the housing casing 404 is made from a vacuum formed process and the sidewalls 414 and top wall 410 are integrally formed from a single sheet of PVC. PETG or other suitable material comprising an opening 412. The plastic material is preferably transparent. However, an opaque or a semi-opaque material may be used without deviating from the spirit and scope of the present disclosure.

In one exemplary embodiment, the dispenser or dispensing opening 412, which is shown with a star like configuration, is located at the center of the top wall 410. However, as described in previous embodiments, the dispensing opening 412 may be located on other appropriate part or location of the dispenser 400 and it may have different opening configurations, such as an oval configuration, a round configuration, or a square configuration.

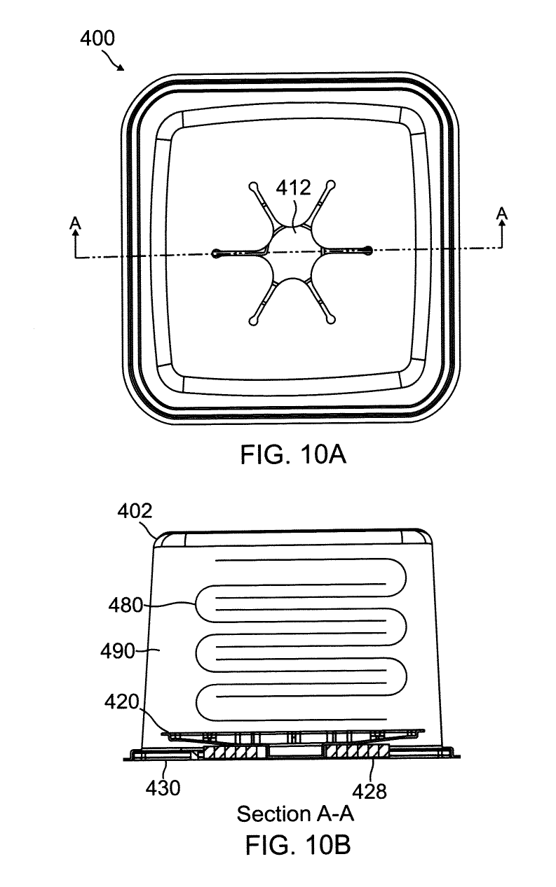

The dispenser 400 further comprises a dispenser base 430. The base 430 comprises a base wall 432 having a recess 434 sized and configured for receiving and centering a biasing member 428, as further discussed below. The recess 434, or at least part of the recess, is solid for supporting the biasing member. In alternative embodiments, the base wall 432 comprises a plurality of recesses configured for receiving and centering a plurality of biasing members, such as for locating two or more biasing members instead of a single biasing member. Together, the top wall 410, the sidewalls 414 and the base wall 432 define a container cavity 490 (FIG. 10B) for receiving a plurality of folded gloves.

Similar to previously described embodiments, the dispenser base 430 and the dispenser top 402 each has a peripheral flange 438, 406 that generally overlap one another and together have a common peripheral edge. However, the edges do not have to form a common edge so long as the two flanges are capable of being attached together. The dispenser base 430, like the casing 402, is made from a plastic vacuum forming process to create a plurality of ribs or ridges for both strengthening the base and for creating the recess area 434 and the flange 438. After filling the container cavity 490 of the housing casing 404 with a stack of gloves 480 (FIG. 10B) folded by a superjacent opposing fold method, the dispenser base 430 is mated with the dispenser top 402 to align the two flanges 406, 438, which are subsequently bonded, glued or welded together to form a sealed dispenser 400. In another example, both the flanges 406, 438 incorporate raised protrusions that extend in the same direction so that when mated, one set of protrusions located on one flange snap fit into the other set of protrusions located on the other flange to snuggly secure the dispenser base to the dispenser top prior to bonding or welding. In one specific example, each flange has twelve spaced-apart protrusions but different numbers of protrusions may be practiced without deviating from the spirit and scope of the present disclosure.

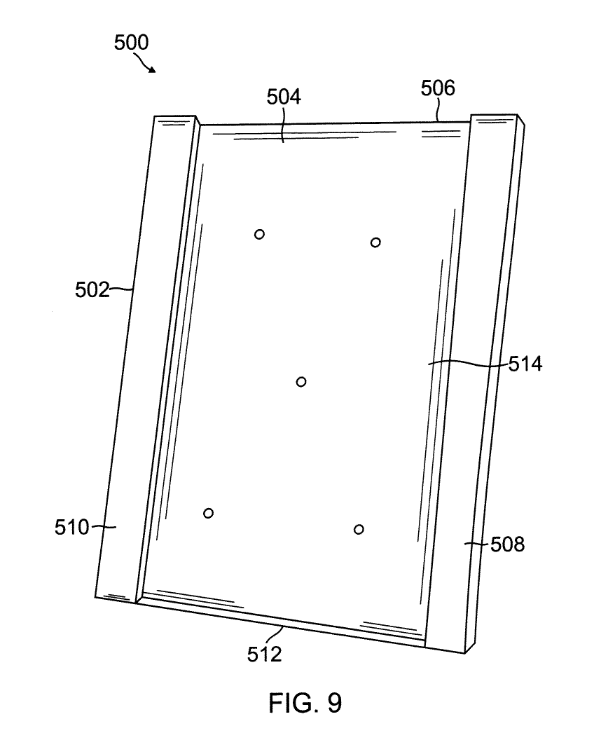

In one embodiment, the dispenser 400 can be mountable on a mounting bracket 500 shown in FIG. 9, which is not drawn to scale. Broadly speaking, the mounting bracket 500 comprises a body 502 configured to accommodate the dispenser 400. The body 502 can include a top surface 504, a bottom surface 506 on the opposite side, two parallel spaced apart retaining channels 508, 510, and a blocking wall 512, which can be a raised lip, another channel, or a straight wall. The channels 508, 510 and the blocking wall 512 define a receiving space 514 for receiving the dispenser 400. In operation, the dispenser 400 can be mounted to the bracket 500 by sliding the welded flanges 406/438 under the retaining channels 508, 510 in a direction parallel to the lengthwise axis of the channels until the dispenser 400 touches or contacts the blocking wall 512. The bracket 500 can include attachment means, such as through bores, for use to mount the bracket to a surface or a workstation, such as to a wall. Alternatively, the dispenser 400 may be used as a freestanding glove dispenser by placing it on a working surface, such as on a shelf, a table, a counter, or a workstation.

Referring again to FIG. 8, in one embodiment, the dispenser 400 further comprises a tray 420 for supporting the stack of gloves within the cavity 490 (FIG. 10B). The tray 420 is movable in a longitudinal direction from the dispenser base 430 towards the dispenser opening 412 with the aid of one or more biasing members 428 and in response to the number of remaining gloves located inside the dispenser, as further discussed below. Thus, the tray 420 is movable relative to the dispenser housing 402. The tray 120 may be made from a semi-rigid transparent thermoplastic material, such as from polyethylene terephthalate (PETG) or PVC material. In alternative embodiments, the tray 420 may be made from cardboard or other non-plastic material. The tray 420 may be formed using a vacuum forming process and comprises a plurality of ribs 422 and a recess area 423 having a centering post 424. In alternative embodiments, the tray 120 comprises a plurality of projections for mating with a plurality of biasing members. The projection(s) or post(s) is configured to project into the center of the biasing member(s), as further discussed below.

With further reference to FIG. 8, the tray 420 may be placed over the biasing member 428 with the centering post 424 projecting into the opening of the biasing member to retain the plate in relative orientation therewith. The biasing member 428 is configured to move the tray 420 in a vertical or longitudinal direction in response to the pressure, weight, and/or physical presence of the stack of gloves located within the dispenser 400. The biasing member 428 moves the tray 420 vertically by exerting a biasing force on the tray 420. The upward vertical movement of the tray 420 allows the next glove to be easily accessible to a user without the user having to reach inside the dispensing opening 412 to retrieve the next glove as the stack of gloves is reduced from usage. Furthermore, it is known from U.S. Pat. Nos. 6,901,723 and 7,063,233 that when one glove is dispensed from a stack of gloves, the cuff section of the next glove to be dispensed is pulled out through the dispenser opening to facilitate removal of that next glove. Such operation may be used herein to facilitate removal of gloves. Furthermore, the present dispenser 400 is susceptible of being used with other types of folding techniques, such as simple folds and placing the gloves on top of one another without inter-mixing in superjacent fold format.

The biasing member 428 can be a spring, a shaped memory foam, a leaf spring, or a low durometer elastomer. In a preferred embodiment, the biasing member 428 is a helical coil spring. In a most preferred embodiment, the biasing member 428 is a conical coil spring. The biasing member 428 can be made from any suitable materials, including without limitations metals and plastics. In one embodiment, the biasing member 428 is made from a glass-reinforced plastic. As an example, the glass-reinforced plastic is an acetal copolymer with greater than 15% by volume reinforced glass. In a specific example, the glass-reinforced plastic includes CELRON® acetal copolymer grade GC25A with 25% reinforced fiber glass. In alternative embodiments, the biasing member 428 may be made from stainless steel. The biasing member 428 may be made by conventional methods that are well known in the art. In one embodiment, the biasing member 428 is made by injection molding. The conical shape spring allows the biasing member 428 to compress nearly or completely flat for accommodating a full stack or a maximum stack of gloves, as shown in FIG. 10B. The spring should be sized with a spring travel and constant to sufficiently bias a stack of 50 to about 80 pairs of gloves that can collectively weigh about 1.3 pounds, such as 1.0 pound to about 1.5 pounds. However, for larger or smaller number of gloves, the spring can be sized to operate accordingly.

In one embodiment, the biasing member 428 comprises a first end 426 and a second end 427. The first end 426 is configured to mate with the centering post 424 on the tray 420, such as receiving the post within the perimeter of the first end. The second end 427 is configured to be placed within the recess 434 of the dispenser base 430. When assembled, as shown in FIG. 10B, the biasing member 428 urges the tray 420 vertically or along a longitudinal direction of the housing in response to the presence and pressure exerted by the stack gloves 480 located on top of the tray 420.

In alternative embodiments, the tray 420 is movable in the upward direction with the aid of a plurality of biasing members 428. The plurality of biasing members 428 are configured at one end to be received by recesses located on the dispenser base 430 and at the other end to engage with projections extending from the tray 420. The plurality of biasing members 428 are distributed such that they exert an even biasing force on the tray 420 and to move the tray upward in response to the number of remaining gloves located inside the dispenser. In one specific embodiment, four biasing members 428 extend from recesses located at four corners of the dispenser base 430 and engage with projections protruding from the corresponding four corners of the tray 420. In another embodiment, the plurality of biasing members 428 are distributed on different parts of the base wall 432 such that they exert a balanced biasing force on the tray 420 to move it in the upward direction.

FIG. 10A is a top view of the dispenser 400 shown in FIG. 8. FIG. 10B is a cross-sectional side view of the dispenser 400 of FIG. 10A taken along line A-A with a stack of gloves 480 shown in schematic. Similar to previously described embodiments, a removable tab (not shown) may be placed over the dispensing opening 412 to seal the opening during packaging, shipping, and/or storage.

The stack of gloves 480, as schematically shown in FIG. 10B, takes up most of the receiving space 490 within the dispenser 400, compresses the biasing member 428, and forces the tray 420 against the dispenser base 430. In this configuration, the biasing member 428 remains in a collapsed state by the presence and pressure of the stack of gloves 480 located on top of the tray 420. As the gloves are dispensed, the stack of gloves gets smaller and the pressure or weight exerted on the tray 420 lessens. In response to this reduced size and weight from the stack of gloves, the biasing member 428 expands and urges the tray 420 and the stack of remaining gloves in an upward direction towards the dispensing opening 412. Thus, regardless whether the dispenser is full or nearly empty, the gloves are always easily accessible due to the biasing force of the biasing member forcing the gloves to the general proximity of the dispensing opening for removal. As clearly shown in FIG. 10B, the biasing member 428 is part of the dispenser 400 and not part of the holder, as shown in FIG. 4B. Thus, the dispenser 400 of the present disclosure incorporates a movable tray that automatically moves relative to the housing to urge the gloves upwardly towards the dispenser opening without a biasing force from an external source.

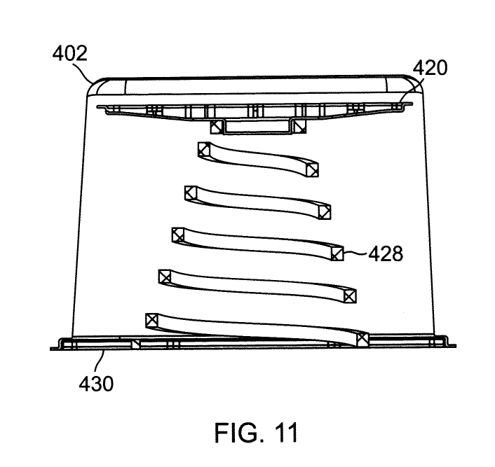

FIG. 11 shows a cross-sectional side view of the dispenser 400 when empty or nearly empty. Without the pressure and presence of the gloves, the biasing member 428 expands to its fully or near fully expanded state to urge the tray 420 towards the dispenser opening 412. In one embodiment, the tray 420 is forced into contact with the top wall 410 when the gloves are emptied. In another example, the tray 420 is spaced apart from the top wall 410 by a small gap when fully emptied, such as by about ⅛-inch to about 1½ inches.

When the dispenser 400 is emptied, it may be disposed of by appropriate means. It should be noted that the use of a plastic biasing member, versus for example a metal biasing member, facilitates the disposal of the dispenser 400 when emptied. For example, for a dispenser containing a metal biasing member, its different components need to be separated and sorted according to the materials that they are made from, since plastics and metals are recycled differently. As such, a further feature of the present dispenser 400 is the ability to be disposed of without the need to first separate its different components. For example, the empty dispenser 400 may be placed in a plastic recyclable bin without first separating it into components. In one embodiment, the dispenser 400 may be reusable by incorporating means for opening the dispenser so that additional gloves may be refilled when the case is low or empty.

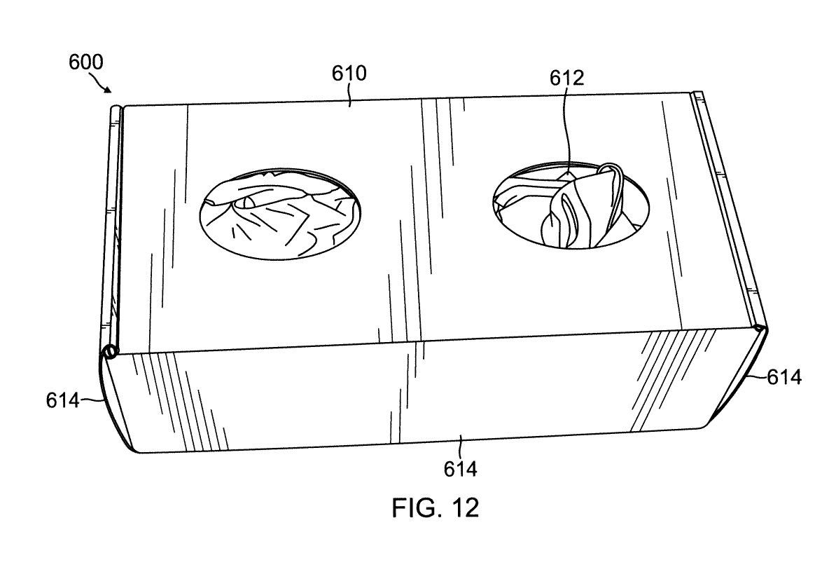

FIG. 12 illustrates a dispensing package 600 provided in accordance with aspects of the present disclosure. In one exemplary embodiment, the dispensing unit 600 is configured to contain two dispensers, such as two dispensers 400 of FIG. 8. In alternative embodiments, the dispensing package 600 is configured to enclose three or more glove dispensers. The dispensing package 600 can have a rectangular, a square, a cylindrical or any other appropriately shaped container configuration to accommodate the two glove dispensers 400, or more glove dispensers as the case may be. The dispensing package 600 may be made from any suitable natural, synthetic, composite, and laminate materials, including, without limitation, paper and board materials; metals and foils; rubber products; and plastics such as cellulose acetate butyrate (CAB), polystyrene, polyethylene terephthalate (PETG); polycarbonate; polyethylene, including PET, HDPE, and LDPE; polytetrafloralethylene; acetal, acrylic and combinations thereof. In a preferred embodiment, the dispensing package 600 is made from cardboard material, such as corrugated fiberboard.

With further reference to FIG. 12, the dispensing package 600 comprises a top wall 610 and a plurality of sidewalls 614. In one exemplary embodiment, the top wall 610 comprises two openings 612 configured to provide access to the dispensing openings 412 of the two enclosed dispensers 400. The openings 612 are shown with an oval configuration in the illustrated embodiment. However, the openings 612 may have different configurations, such as a round configuration, a star configuration or a square configuration.

In one embodiment, a removable tab (not shown) is placed over each of the openings 612 to seal the openings during packaging, shipping and storage. In alternative embodiments, the openings 612 comprise pierceable tabs (not shown) integrally formed with the openings 612 and separated from the openings 612 by perforated lines. To expose the openings 612, the pierceable tabs can be removed by pressing down on the perforated lines to detach the tabs from the openings 612.

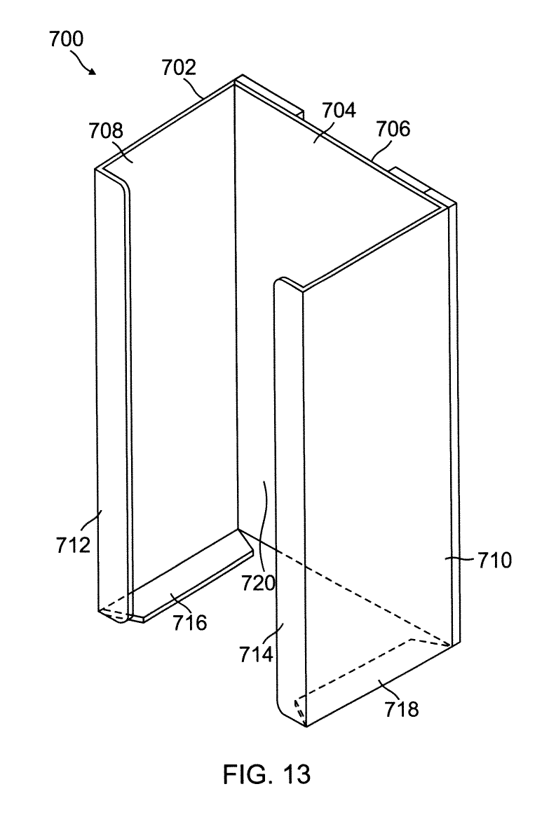

In some embodiments, the dispensing package 600 is mountable on a mounting bracket 700 shown in FIG. 13. The bracket 700 may be made from any suitable natural, synthetic, composite, and laminate materials, including, without limitation; metals; rubber products; and plastics such as cellulose acetate butyrate (CAB), polystyrene, polyethylene terephthalate (PETG); polycarbonate; polyethylene, including PET, HDPE, and LDPE; polytetrafloralethylene; acetal, acrylic and combinations thereof. The bracket 700 comprises a body 702 configured for accommodating the package 600. The body 702 can include a top surface 704, a bottom surface 706 on the opposite side and two parallel spaced apart lateral panels 708, 710. The lateral panels 708. 710 each comprises a front edge flange 712 and 714, respectively. The lateral panels 708, 710 further comprise bottom edge support flanges 716 and 718, respectively. The top surface 704, the lateral panels 708, 710 and the flanges 712, 714, 716 and 718 define a receiving space 720 for receiving the dispensing package 600. In an alternative embodiment, a third lateral panel is used instead of two bottom support flanges 712, 718. Also, the bracket 700 may be mounted in various orientations rather than for vertical installation and removal, as shown.

In operation, the package 600 can be mounted onto the bracket 700 by sliding the package 600 under the lateral panels 708 and 710 until the dispensing package 600 touches or contacts the bottom edge support flanges 716 and 718. The flanges 712, 714, 716 and 718 support the dispensing package 600 and prevents it from falling downwardly or forwardly from the bracket. The bracket 700 can include attachment means, such as through bores or magnetic pads affixed to the bottom surface 706, for use to mount the bracket to a surface or a workstation. Alternatively, the package 600 may be used as a freestanding glove dispenser by placing the dispensing package on a working surface, such as a table or a shelf. Still alternatively, the bracket 700 may incorporate retaining channels for retaining the edges of the flange of the dispenser package 600 rather than for retaining the housing walls.

Accordingly, an aspect of the present disclosure is understood to include a dispenser having a biasing member for urging a bundle gloves towards an opening on a dispenser. A further aspect of the present disclosure is a dispenser comprising a housing for containing a plurality of gloves having a movable tray that moves relative to the housing to move the plurality of gloves toward the opening on the housing when the tray is urged by a biasing force. A still further aspect of the present disclosure is a dispensing package for housing two or more glove dispensers; each glove dispenser having a biasing member for urging a bundle gloves towards an opening on a dispenser. A still further aspect of the present disclosure is a method for forming a combination dispenser and plurality of gloves wherein the dispenser comprises a tray for supporting a bundle of gloves and wherein the tray is movable relative to the housing when urged by a biasing member, such as by a helical spring. Another feature of the present disclosure is a provision for disposing the dispenser without first separating its components. Yet, a still further feature of the present disclosure is a dispenser comprising a housing and a base and wherein a spring and a tray located within the housing cavity are movable from the base towards an opening located on a top wall of the housing. A specific feature of the present apparatus, device and method is the use of a glass-filled thermoplastic material to make a conical spring.

Another feature of the present disclosure is understood to include a spring made from a glass reinforced thermoplastic material having an outer coil of a first diameter and an inner coil of a smaller diameter. Wherein the outer coil is configured to urge against a base and the inner coil is configured to urge against a tray having a plurality of gloves placed thereon.

As used herein, the terms first, second, top, side, upper, lower, back, etc. are for reference purposes only and are not structurally limiting. For example, the term top wall is relative and may be considered a sidewall or bottom wall in different contexts.

Many alterations and modifications may be made by those having ordinary skill in the art, without departing from the spirit and scope of the present disclosure. For example, features and aspects specifically discussed for one embodiment but not another may be interchangeable provided the modification does not conflict or made inoperable. Therefore, it must be understood that the illustrated embodiments have been set forth only for the purposes of examples, and that the embodiments should not be taken as limiting the disclosure as defined by the following claims. The following claims are, therefore, to be read to include not only the combination of elements which are literally set forth, but all equivalent elements for performing substantially the same function in substantially the same way to obtain substantially the same result. The claims are thus to be understood to include those that have been illustrated and described above, those that are conceptually equivalent, and those that incorporate the ideas of the present disclosure.

What is claimed is:

1. A dispenser adapted to store gloves, the dispenser comprising:
a dispenser housing comprising a plurality of side walls, a top wall comprising an opening and a height along a longitudinal direction;
a dispenser base comprising a base wall; wherein the side walls, the top wall and the base wall define a receiving space for receiving a bundle of gloves;
a tray movable along the longitudinal direction located within the receiving space and positioned between the dispenser base and the top wall; and
at least one biasing member for biasing the tray to move along the longitudinal direction relative to the dispenser base towards the top wall.

2. The dispenser of claim 1, wherein the at least one biasing member is a spring, a shaped memory foam, a leaf spring, or a low durometer elastomer.

3. The dispenser of claim 1, wherein the at least one biasing member is a conical coil spring.

4. The dispenser of claim 1, wherein the base wall comprises at least one recess for receiving a first end of the at least one biasing member.

5. The dispenser of claim 1, wherein the tray comprises at least one protrusion for engaging a second end of the at least one biasing member.

6. The dispenser of claim 1, wherein the dispenser is located in a receiving space of a separately formed holding device.

7. The dispenser of claim 1, wherein the dispenser housing comprise a first flange and the dispenser base comprises a second flange.

8. The dispenser of claim 1, wherein the at least one biasing member projects through the dispenser base to contact the tray.

9. A combination glove dispenser and gloves, comprising:
a glove bundle having a predetermined number of gloves disposed in a dispenser housing; the dispenser housing comprising a dispenser top, a plurality of sidewalls and a base wall defining a receiving space;
a tray movable within the receiving space and positioned between the base wall and the dispenser top; and
a projection formed on the tray for engaging the tray to a biasing member.

10. The combination of claim 9, further comprising a biasing member and wherein the biasing member is located in the receiving space between the base and the dispenser top.

11. The combination of claim 9, further comprising a biasing member and wherein the biasing member includes a spring, a shaped memory foam, a leaf spring, or a low durometer elastomer.

12. The combination of claim 9, further comprising a conical spring and wherein the conical spring is made from glass-reinforced plastic.

13. The combination of claim 12, wherein glass-reinforced plastic is an acetal copolymer with greater than 15% by volume reinforced glass.

14. The combination of claim 13, wherein the glass-reinforced plastic includes acetal copolymer grade with 25% reinforced fiber glass.

15. The dispenser of claim 9, wherein the plurality of side walls comprise a first flange, the base wall comprises a second flange; and wherein the first flange and the second flange are welded together.

16. A method for forming a combination dispenser and plurality of gloves, the method comprising:
forming a dispenser body comprising a plurality of sidewalls, a top wall having a dispensing opening and an installation opening;
placing a plurality of gloves through the installation opening into a interior space of the dispenser body;
positioning a tray subjacent the plurality of gloves, the tray adapted to move within the cavity:
providing a biasing member for biasing the tray to move in a longitudinal direction towards the top wall; and
closing the installation opening with a base wall.

17. The method of claim 16, further comprising welding a first flange on the base wall with a second flange on the dispenser body.

18. The method of claim 16, wherein the plurality of side walls and the top wall are integrally formed.

19. The method of claim 16, wherein the biasing member includes a spring, a shaped memory foam, a leaf spring, or a low durometer elastomer.

20. The method of claim 16, further comprising engaging the combination dispenser and plurality of gloves to a holding member.

21. A combination glove dispenser and gloves, comprising:
a dispenser case comprising a plurality of walls defining a receiving space adapted to contain a bundle of gloves, the dispensing case comprising an opening for dispensing the gloves received within the receiving space.
